(19) United States
(12) Patent Application Publication
(10) Pub. No.: US 2025/0032816 A1
(43) Pub. Date: Jan. 30, 2025

(54) INTRAORAL PHOTOTHERAPY PROBE

(71) Applicant: Mureva Phototherapy Inc., Strongsville, OH (US)

(72) Inventors: Vedang KOTHARI, San Diego, CA (US); Jordan W. OJA, Stow, OH (US); Jason D. LAZZARA, Brooklyn Heights, OH (US); Samuel J. SHELNUTT, Olmsted Township, OH (US)

(73) Assignee: Mureva Phototherapy Inc., Strongsville, OH (US)

(21) Appl. No.: 18/359,430

(22) Filed: Jul. 26, 2023

(51) Int. Cl. A61N 5/06

(52) U.S. Cl. CPC A61N 5/0603; A61N 2005/0606; A61N 2005/0632; A61N 2005/065

(57) ABSTRACT

A phototherapy device is provided for delivering targeted phototherapy to an oral cavity. The phototherapy device includes a main body configured to be received within the oral cavity, a light source for emitting light, and an air gap located between the light source and a shell for improved light delivery to the pharyngeal tissues of the oral cavity.

### TECHNICAL FIELD

The present disclosure relates generally to phototherapy and more particularly to an intraoral phototherapy probe.

### BACKGROUND

Phototherapy has been recognized as an effective treatment method for various conditions, including Oral Mucositis (OM), providing pain relief and promoting healing. Phototherapy can be administered through different modalities, such as Low-Level Laser Therapy (LLLT) or Light Emitting Diode (LED) arrays, both of which deliver light energy directly to the target tissue.

Currently, there are two primary methods for delivering phototherapy for the treatment of oral conditions, including Oral Mucositis (OM): low-level laser therapy and light emitting diode (LED) arrays. Oral Mucositis is a common and highly significant side effect of cancer therapy, causing significant discomfort and pain for affected patients.

Despite the potential benefits of low-level laser therapy, there are several barriers to its widespread acceptance and use. These include the high cost of laser equipment, the labor-intensive nature of the treatment, interoperator variability, and the requirement for specialized training. Moreover, patients receiving this form of treatment often experience discomfort and pain due to the need to keep their mouths open for extended periods of time, which can be particularly challenging as the Mucositis progresses.

### SUMMARY

The present disclosure provides a phototherapy device for delivering targeted phototherapy to the oral cavity, comprising a main body configured to be received within the oral cavity, a light source supported by the main body for illuminating pharyngeal tissues, and an air gap between the light source and a protective shell for enhanced light delivery to the target tissues.

The invention described in this application addresses the limitations of existing phototherapy methods by providing a novel phototherapy device specifically designed for the efficient, comfortable, and targeted treatment of oral conditions, such as Oral Mucositis. This innovative device delivers light energy directly to the affected tissues within the oral cavity in a more user-friendly, accessible, and customizable manner.

Through this innovative phototherapy device, patients suffering from oral conditions, such as Oral Mucositis, can experience more efficient, comfortable, and targeted treatments, significantly improving their quality of life during the healing process. The various embodiments and features of the invention, as described herein and illustrated in the drawings, highlight the potential of the device to revolutionize the field of oral phototherapy and provide a more accessible and user-friendly treatment option for a wide range of patients.

While a number of features are described herein with respect to embodiments of the invention; features described with respect to a given embodiment also may be employed in connection with other embodiments. The following description and the annexed drawings set forth certain illustrative embodiments of the invention. These embodiments are indicative, however, of but a few of the various ways in which the principles of the invention may be employed. Other objects, advantages, and novel features according to aspects of the invention will become apparent from the following detailed description when considered in conjunction with the drawings.

### BRIEF DESCRIPTION OF THE DRAWINGS

The annexed drawings, which are not necessarily to scale, show various aspects of the invention in which similar reference numerals are used to indicate the same or similar parts in the various views.

FIG. 1 is a schematic view of an exemplary embodiment of a phototherapy device including a main body having a shell for maintaining an air gap.

FIG. 2 is a schematic view of an exemplary embodiment of a phototherapy device including a light source.

FIG. 3 is a side view of an exemplary embodiment of a phototherapy device having an articulable main body.

FIG. 4 is a side view of an exemplary embodiment of a phototherapy device having a main body with two points of articulation.

FIG. 5 is a side view of an exemplary embodiment of a phototherapy device having a deformable main body.

FIG. 6 is a schematic view of an exemplary embodiment of a phototherapy device including a camera.

FIG. 7 is a side view of an exemplary embodiment of a phototherapy device having an articulable main body and a camera.

FIG. 8 is a side view of an exemplary embodiment of a phototherapy device having a main body with two points of articulation and a camera.

FIG. 9 is a schematic view of an exemplary embodiment of a phototherapy device having a main body and side wings.

FIG. 10 is a side view of the phototherapy device of FIG. 9 with the side wings outlined.

FIG. 11 is a top view of an exemplary embodiment of a phototherapy device having an internal breathing tube and a protrusion.

FIG. 12 is a side view of the phototherapy device of FIG. 11.

FIG. 13 is a top view of an exemplary embodiment of a phototherapy device having an external breathing tube and a protrusion.

FIG. 14 is a side view of the phototherapy device of FIG. 13.

FIG. 15 is a bottom view of an exemplary embodiment of a phototherapy device having tongue protrusions.

FIG. 16 is a side view of the phototherapy device of FIG. 15.

FIG. 17 is a side view of a phototherapy device having a main body portion including an upper portion and a lower portion located within the oral cavity.

FIG. 18 is a top view of the phototherapy device of FIG. 17 located within the oral cavity.

FIG. 19 is a front view of the phototherapy device of FIG. 17.

FIG. 20 is a top view of the phototherapy device of FIG. 17.

FIG. 21 is a side view of the phototherapy device of FIG. 17.

The present invention is described below in detail with reference to the drawings. In the drawings, each element with a reference number is similar to other elements with the same reference number independent of any letter designation following the reference number. In the text, a reference number with a specific letter designation following the reference number refers to the specific element with the number and letter designation and a reference number without a specific letter designation refers to all elements with the same reference number independent of any letter designation following the reference number in the drawings.

### DETAILED DESCRIPTION

The present invention relates generally to a phototherapy device for providing targeted phototherapy to an oral cavity. In particular, the invention pertains to a phototherapy system with a main body configured to be received within the oral cavity, a light source for emitting light, and an air gap located between the light source and a shell for improved light delivery to the pharyngeal tissues of the oral cavity.

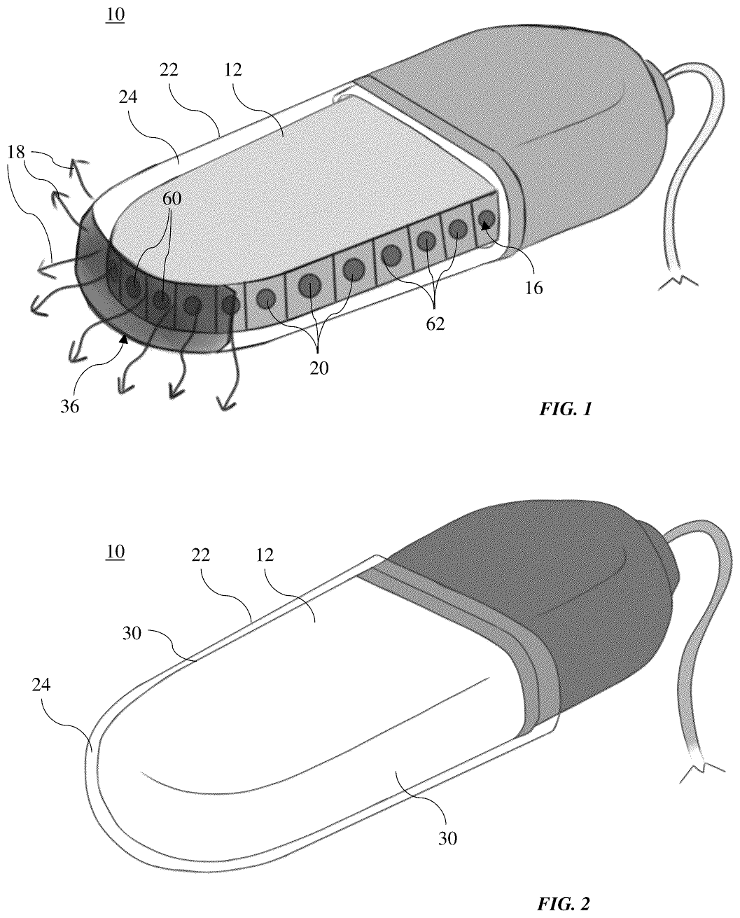

In one embodiment of the invention depicted in FIGS. 1 and 2, the phototherapy device 10 includes a main body 12 configured to be received within the oral cavity 14 of a user. The main body 12 may be designed to fit comfortably and securely in the oral cavity 14, with an ergonomic shape to minimize discomfort and prevent accidental dislodging during use.

The phototherapy device 10 further includes a light source 16 configured to emit light 18 (also referred to as electromagnetic radiation) and mechanically supported by the main body 12. The light source 16 may include one or more light emitters 20, such as light-emitting diodes (LEDs) or laser diodes, which generate the light 18 at specific wavelengths suitable for phototherapy applications. The light source 16 may be powered by a battery (e.g., housed within the main body 12) or external power supply connected to the device (not shown).

When the main body 12 is positioned within the oral cavity 14, the light source 16 is located within the oral cavity 14 such that the emitted light 18 illuminates the pharyngeal tissues of the oral cavity 14. The light source 16 may be positioned, angled, or focused in a manner that directs the emitted light 18 towards the target tissues for phototherapy treatment.

In a further aspect of the invention, the main body 12 of the phototherapy device 10 includes a shell 22 separating the light source 16 from the oral cavity 14. The shell 22 may be composed of a transparent or semi-transparent material that permits the passage of the emitted light 18 while providing a barrier (e.g., mechanical barrier) between the light source 16 and the oral cavity 14. The barrier provided by the shell 22 helps protect the light source 16 from contamination and the user from any potential harm that could result from direct contact with the light source 16.

Additionally, the phototherapy device incorporates an air gap 24 located between the light source 16 and the shell 22. The air gap 24 enhances the delivery of emitted light 18 to the pharyngeal tissues by mitigating the absorption or scattering of the light 18 by the shell 22 or other components (e.g., coatings, etc.) within the device 10. The air gap 24 bolsters the efficiency of phototherapy treatment by ensuring that a larger percentage of the emitted light reaches the intended target tissues of the oral cavity 14.

The phototherapy device 10 may include structures positioned between the shell 22 and an interior surface of the main body 12 to maintain the air gap 24 located between the light source 16 and the shell 22. The structures positioned between the shell 22 and the interior surface of the main body 12 can take various forms and configurations. In one embodiment, the structures can be elongated, rigid members that are positioned at regular intervals around the circumference of the main body 12.

When the main body 12 of the phototherapy device 10 is deformable, as shown in FIGS. 8 and 10, the structures play an important role in maintaining the air gap 24 between the shell 22 and the light source 16. The deformable nature of the main body 12 can cause the shell 22 to move closer to or further away from the light source 16, depending on the position of the main body 12. This movement can result in changes to the size and shape of the air gap 24, potentially affecting the efficiency of light delivery to the pharyngeal or oropharyngeal tissues.

To address this issue, the structures positioned between the shell 22 and the interior surface of the main body 12 are designed to maintain a consistent distance between the shell 22 and the light source 16. This helps to ensure that the air gap 24 exists regardless of the shape or position of the main body 12.

The structures can also be designed to allow for some flexibility in the main body 12, while still maintaining the air gap 24. For example, the structures can be attached to the interior surface of the main body 12 using flexible materials such as rubber or silicone. This allows the main body 12 to deform and move while still maintaining the air gap 24 and ensuring efficient light delivery to the intended target tissues.

As described above, the air gap 24 located between the light source 16 and the oral cavity 14 acts to avert light scattering and to reduce optical losses. When light traverses through various media (e.g., from the light source, through a solid surface, and directly into the oral cavity), a fraction of the light may be scattered or reflected due to the disparity in refractive indices between the different media. This can result in diminished light intensity and reduced effectiveness in targeting the desired tissues.

By incorporating an air gap 24, the number of transitions between different media is reduced, thereby decreasing the likelihood of light scattering and optical losses. This ensures a more consistent and efficient delivery of light to the pharyngeal or oropharyngeal tissues targeted by the phototherapy device 10.

In one embodiment, the shell 22 of the phototherapy device is a rigid shell specifically designed to maintain the air gap 24 by preserving a separation between the shell and the light source. In this way, the shell 22 may ensure a separation (e.g., of a minimum distance) between the light source 16 and the shell 22 is consistently maintained, enhancing the efficiency of light delivery to the target tissues, e.g., while additionally providing protection to the oral cavity 14 from direct contact with the light source 16.

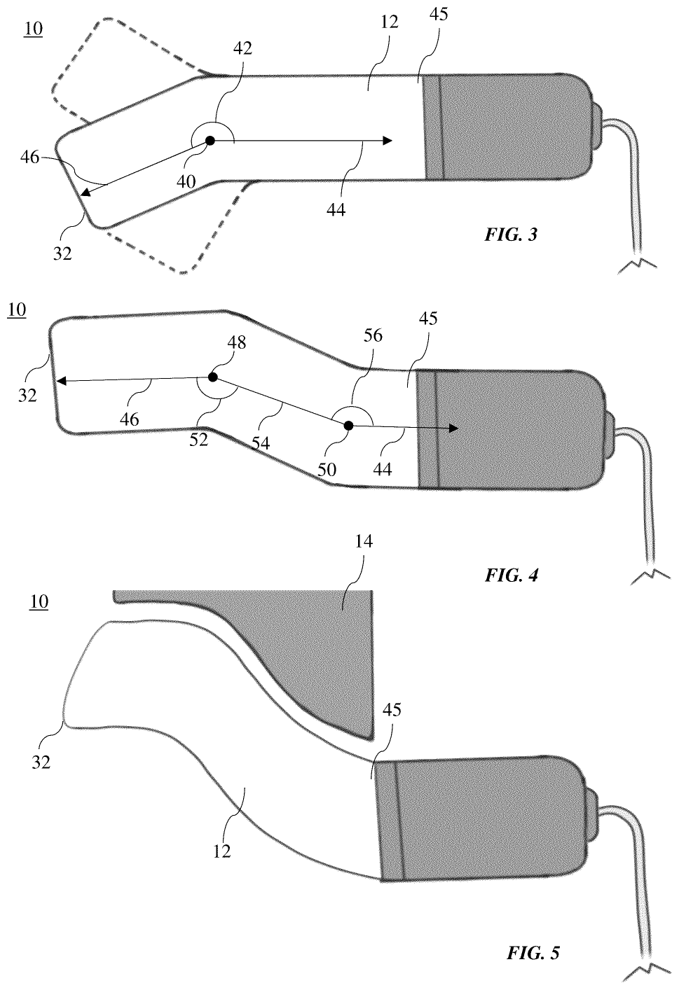

In the embodiment depicted in FIG. 3, the main body 12 of the phototherapy device 10 is articulable along a point of articulation 40, forming an angle of articulation 42 between a proximal plane 44 and a distal plane 46 of the main body 12. This allows a portion of the emitted light 18 to be targeted onto the pharyngeal tissues more effectively. The proximal plane 44 extends between the lateral walls 30 of the main body 12, a proximal end 45 of the main body 12, and the point of articulation 40, while the distal plane 46 extends between the lateral walls 30, the distal end 32 of the main body 12, and the point of articulation 40. The articulation provided by the point of articulation 40 may enhance the adaptability of the device 10 to accommodate various oral cavity shapes and sizes, ensuring optimal light delivery to the target tissues of the oral cavity 14.

In the embodiment shown in FIG. 4, the main body 12 of the phototherapy device 10 is articulable along both a distal point of articulation 48 and a proximal point of articulation 50, enabling more precise targeting of the emitted light onto the pharyngeal tissues. The distal point of articulation 48 forms a distal angle of articulation 52 between a central plane 54 of the main body 12 and a distal plane 46 of the main body 12, while the proximal point of articulation 50 forms a proximal angle of articulation 56 between the central plane 54 and the proximal plane 44 of the main body 12. The proximal plane 44 may extend between lateral walls 30 of the main body 12, a proximal end 45 of the main body 12, and the proximal point of articulation 50. The central plane 54 may extend between the lateral walls 30 of the main body 12, the distal point of articulation 48, and the proximal point of articulation 50. The distal plane 46 may extend between the lateral walls 30 of the main body 12, the distal end 32 of the main body 12, and the distal point of articulation 48. This dual articulation enabled by the proximal point of articulation 50 and distal point of articulation 48 may provide even greater adaptability, allowing the device 12 to conform to a wider range of oral cavity shapes for improved phototherapy effectiveness.

Turning to FIG. 5, the main body 12 of the phototherapy device may be made of a memory-forming material that is conformable to the shape of the oral cavity 14 when positioned within it. This feature allows for a comfortable and secure fit, adapting to the unique contours of the user's oral cavity.

As described above, the light source 16 may include light emitters 20 for generating the emitted light 18. The light emitters 20 may include distal light emitters 60 and lateral light emitters 62. The distal light emitters 60 may be positioned along the distal end 32 of the main body 12, such that the light 18 emitted by the distal light emitters 60 illuminates the pharyngeal tissues when the main body 12 is positioned within the oral cavity 14. The lateral light emitters 62, may be positioned along the lateral walls 30 of the main body 12, such that light 18 emitted by the lateral light emitters 62 illuminate the buccal tissues of the oral cavity 14 when the main body 12 is positioned within the oral cavity 14. This arrangement of light emitters 20 may provide comprehensive and targeted illumination of the various tissues within the oral cavity 14, enhancing the overall efficacy of the phototherapy device.

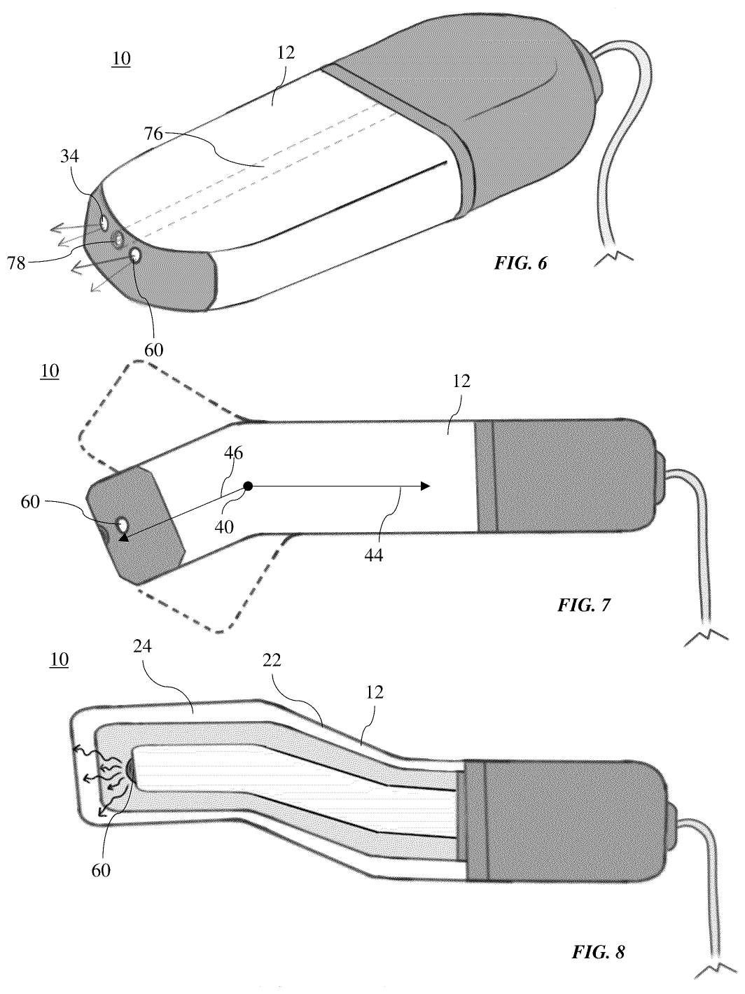

In the embodiment shown in FIGS. 6-8, the main body 12 of the phototherapy device 10 includes lateral walls 30 and a distal end 32 situated between the lateral walls 30. A camera 34 may be located along the distal end 32 and may be configured to image the tissues of the oral cavity 14 being illuminated by the emitted light 18 when the main body 12 is inserted into the oral cavity. That is, the camera 34 may enable visual monitoring of the phototherapy treatment being applied via the phototherapy device, allowing for adjustments or optimization of the treatment parameters, positioning of the main body 12 within the oral cavity 14, or shape of the main body 12 as needed, as well as providing valuable visual feedback to a healthcare provider.

In one embodiment, the distal end 32 is alternatively or additionally equipped with lensing 36 designed to alter the trajectory of the emitted light 18, ensuring that the pharyngeal tissues are effectively illuminated by the emitted light 18. The lensing 36 may aid in optimizing the distribution of the emitted light 18 within the oral cavity, directing the light 18 more precisely toward the target tissues and further improving the overall efficiency and effectiveness of the phototherapy treatment.

The main body of the phototherapy device 12 may include location features, such as irregularities in the surface of the main body. These location features are designed to engage with at least one of the teeth, lips, or palate when the main body is positioned within the oral cavity. This interaction helps ensure that the main body is placed in a particular position and orientation within the oral cavity, promoting consistent and accurate treatment.

Turning to FIGS. 13 and 14, the proximal end 45 of the main body 12 may include a top bite location 64 along a top surface 66 and a bottom bite location 68 along a bottom surface 70. The top bite location 64 may be positioned to interact with top teeth of the oral cavity 14, while the bottom bite location 68 may be positioned to interact with bottom teeth of the oral cavity 14 when the main body 12 is positioned within the oral cavity 14. This configuration may help to stabilize and secure the device within the oral cavity 14 during treatment.

The light emitted 18 from the light source 16 via the lateral walls 30 of the main body 12 may illuminate the buccal tissues of the oral cavity 14 through an opening formed between the upper and lower teeth due to the main body being located between the upper and lower teeth. This may enable effective illumination of buccal tissues, enhancing the overall effectiveness of the phototherapy treatment.

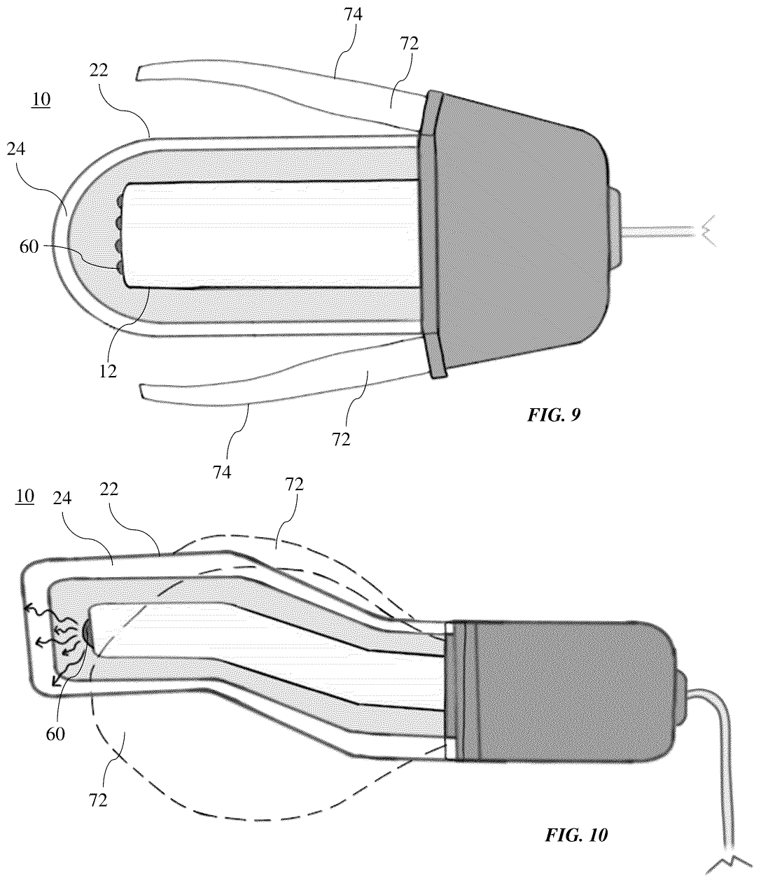

Turning to FIGS. 9 and 10, the phototherapy device 10 further includes side wings 72 adjacent to the lateral walls 30 of the main body 12. Each side wing 72 may include a light-emitting surface 74 configured to emit light 18. When the main body 12 is positioned within the oral cavity 14, the side wings 72 may be strategically placed between the teeth and buccal tissues of the oral cavity 14, with the light-emitting surface 74 facing the buccal tissues. The light 18 emitted from the light-emitting surface 74 may illuminate the buccal tissues, providing targeted phototherapeutic treatment of the buccal tissues.

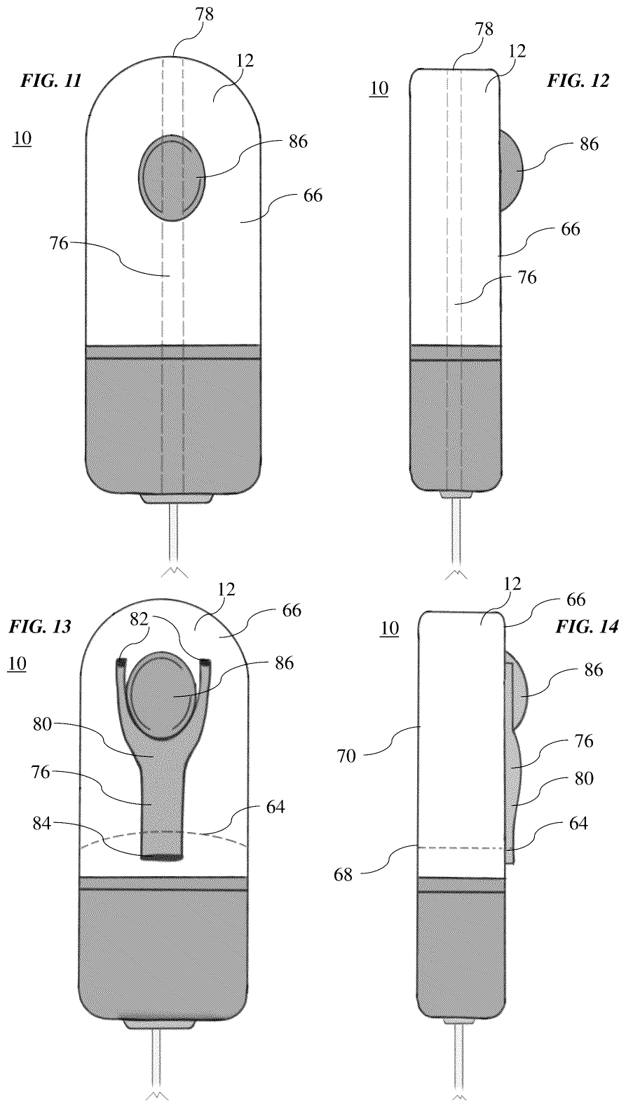

In the embodiment shown in FIGS. 11 and 12, the main body 12 of the phototherapy device 10 includes a breathing channel 76 with an opening 78 extending between the distal end 32 and proximal end 45 of the main body 12. When the device 10 is positioned within the oral cavity 14, the breathing channel 76 may allow for the exchange of air between the oral cavity 14 and an environment external to the oral cavity 14, ensuring user comfort and maintaining unobstructed airflow during treatment.

In the embodiment shown in FIGS. 13 and 14, the phototherapy device 12 includes a breathing apparatus 80 mechanically supported by an exterior surface of the main body 12. The breathing channel may include an oral cavity opening 82, an external environment opening 84, and a breathing channel 76 extending between the two openings 82, 84. When the device 10 is positioned within the oral cavity 14, the breathing channel 76 may allow for air exchange between the oral cavity and the external environment, enhancing user comfort and maintaining proper airflow throughout the phototherapy session.

With continued reference to FIGS. 13 and 14, the main body 12 may also include a protrusion 86 extending from the top surface 66. When the main body 12 is positioned within the oral cavity 14, the protrusion 86 is configured to interact with a roof of the oral cavity 14, reducing the gag reflex that may be caused by the presence of the main body 12 within the oral cavity 14.

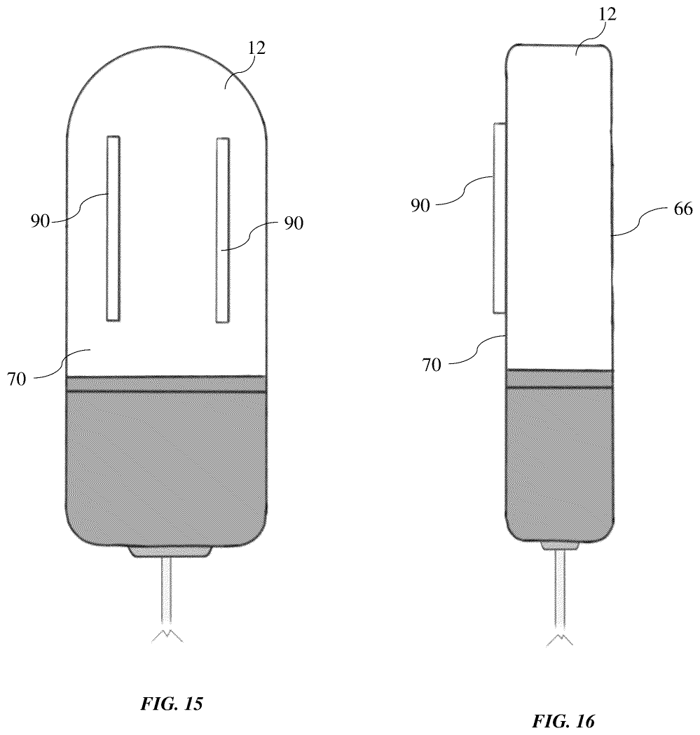

Turning to FIGS. 15 and 16, the main body 12 may include tongue protrusions 90 extending from the bottom surface 70. When the main body 12 is positioned within the oral cavity 14, the tongue protrusions 90 are configured to help depress the tongue 100, helping ensure proper positioning and stabilization of the device during treatment.

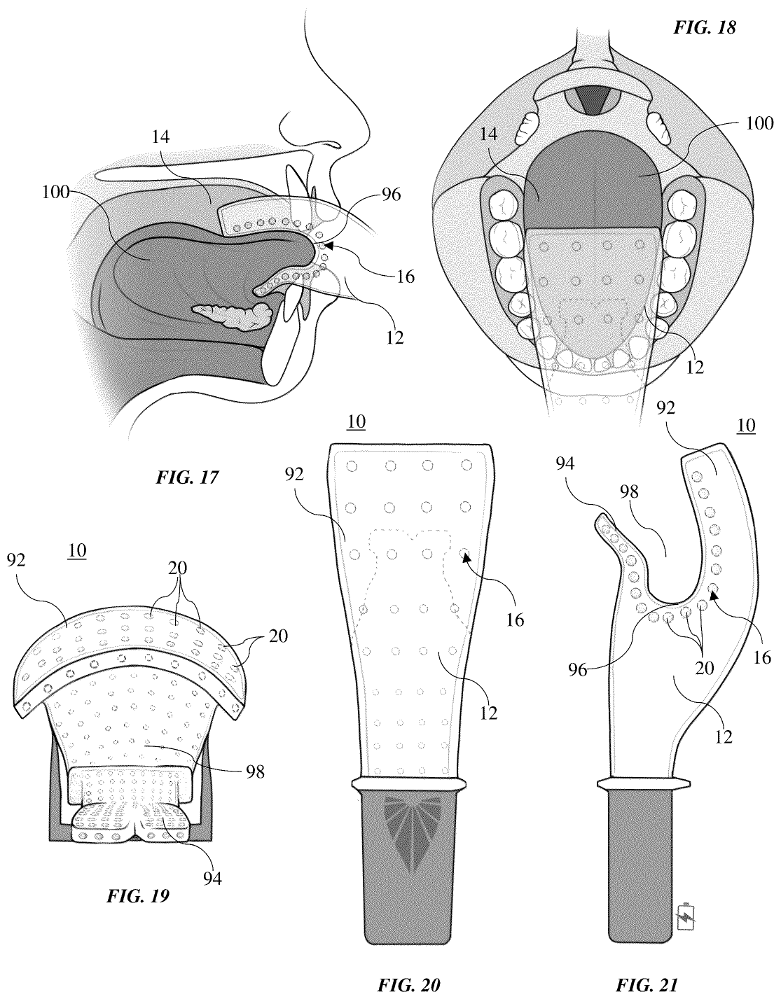

Turning to FIGS. 17-21, the main body 12 includes an upper portion 92, a lower portion 94, and a connecting portion 96 that joins them together. The shape of the upper portion 92, lower portion 94, and connecting portion 96 form a channel 98 shaped to accommodate the insertion of the tongue 100.

As shown in FIGS. 17 and 18, when the main body 12 is properly positioned within the oral cavity 14, the upper portion 92 may overlie the tongue 100, while the lower portion 94 underlies it. The light emitted from the upper portion may target the dorsum of the tongue 100, while the light emitted from the lower portion may illuminate the ventral surface of the tongue 100. As shown in FIGS. 19-21, the main body 12 may include light emitters 20 for illuminating the tongue (e.g., dorsum, ventral surface, and lateral surfaces), and the roof of the mouth.

The phototherapy device may also include processor circuitry mechanically supported by the main body. The processor circuitry may control the emission of light by the light source, allowing for precise and customizable treatment parameters. This feature enables the user or medical professional to tailor the phototherapy session to the specific needs of the individual patient.

The light source may be at least one of a light emitting diode (LED) array, a single high-powered LED, a fiber optic transmitting light, a fiber optic cable, microLEDs, or a laser. As described above, the light source may be located in an enclosure separate from or attached to the probe or the light source may be operatively mounted to the probe, such that the light source is located within the oral cavity when the probe is inserted into the oral cavity.

The light source and light emitters may emit any suitable wavelength of electromagnetic radiation, such as a wavelength from 600 nm to 1000 nm. For example, the light source may emit electromagnetic radiation having a wavelength approximately equal to at least one of 630 nm, 660 nm, 670 nm, 810 nm, or 880 nm. In one embodiment, the light source may emit both therapeutic light and infrared or near infrared light, such that penetration of the therapeutic light into tissues of the oral cavity is improved. That is, the infrared or near infrared light may improve tissue penetration of the therapeutic light. For example, the light source may emit light having a wavelength known to kill bacteria in the mouth.

The main body may be made of any suitable material. In one embodiment, a portion of the main body is made from a soft and/or flexible optically transparent material having a shore A durometer of 60 or less and a percent elongation of greater than 100%. For example, the surface of the main body may have a shore A durometer of 60 or less and a percent elongation of greater than 100%. In an embodiment, the probe is made of at least one of acrylic, glass, silicone, or a polymeric material. As an example, the main body may be made of different formulations of polycarbonate, polymethyl methacrylate, polystyrene, nylon, acrylonitrile butadiene styrene, polyolefin, or other biocompatible thermoplastic elastomer formulations.

The regions of the oral cavity targeted by the light emitted by the phototherapy device 10 may include at least one of the tongue, mandibular and maxillary buccal surfaces of the oral cavity, the floor and roof of the oral cavity, and tonsillar tissues. In one embodiment, the targeted regions of the oral cavity include the tongue, mandibular and maxillary buccal surfaces of the oral cavity, the floor and roof of the oral cavity, and tonsillar tissues. The phototherapy device may apply phototherapy unevenly across the tissues by varying the optical dose received by different tissues. This variation in optical dose may be due to present medical issues in these tissues (e.g., lesions, sores, etc.). Alternatively, this variation may be due to a statistically higher likelihood of these tissues experiencing such issues. In this example, phototherapy may be used as a preventative treatment.

That is, the particular dose of optical power delivered to the target regions may vary between at least two of the target regions. For example, the target regions may include at least one of: a tonsillar region, buccal tissues of an oral cavity, a hard palate, a soft palate, or the tongue. The particular dose of optical power delivered to the different tissues may be varied based on known effective optical doses for treating different issues. For example, the particular dose for the tonsillar region may be different from the particular dose of optical power delivered to the hard palate.

The particular dose of optical power for each of the target regions may be between 10 milliwatts/cm2 and 150 milliwatts/cm2. The particular dose of optical power received by each of the target regions may not vary between the target regions by more than 20%.

The property of light altered by the processor circuitry may include at least one of: an intensity, a wavelength, a duration of emission, a coherence, time modulation of emission, or a distance of emission from the target regions.

The intraoral phototherapy device may be used in a number of applications, several examples of which include oral mucositis, acute necrotizing ulcerative gingivitis (ANUG), periodontal diseases, trismus, decreasing recovery time from oral surgery, light delivery for orthodontics, and photodynamic light therapy, e.g., to activate a chemical mouthwash.

The processor circuitry may have various implementations. For example, the processor circuitry may include any suitable device, such as a processor (e.g., CPU), programmable circuit, integrated circuit, memory and I/O circuits, an application specific integrated circuit, microcontroller, complex programmable logic device, other programmable circuits, or the like. The processor circuitry may also include a non-transitory computer readable medium, such as random-access memory (RAM), a read-only memory (ROM), an erasable programmable read-only memory (EPROM or Flash memory), or any other suitable medium. Instructions for performing the method described below may be stored in the non-transitory computer readable medium and executed by the processor circuitry. The processor circuitry may be communicatively coupled to the computer readable medium and a network interface through a system bus, mother board, or using any other suitable structure known in the art. The processor circuitry may receive parameters for controlling the light source via the network interface. Alternatively or additionally, the processor circuitry may receive particular optical doses for different tissues from the network interface and the processor circuitry may control the light source so that the received optical doses are received by the respective tissues.

All ranges and ratio limits disclosed in the specification and claims may be combined in any manner. Unless specifically stated otherwise, references to “a,” “an,” and/or “the” may include one or more than one, and that reference to an item in the singular may also include the item in the plural.

Although the invention has been shown and described with respect to a certain embodiment or embodiments, equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A phototherapy device for delivering phototherapy to an oral cavity, the phototherapy device including:
a main body configured to be receiving within the oral cavity;
a light source configured to emit light and mechanically supported by the main body, such that when the main body is positioned within the oral cavity the light source is located within the oral cavity and the emitted light illuminates pharyngeal tissues of the oral cavity;
wherein the main body includes:
a shell separating the light source from the oral cavity;
an air gap located between the light source and the shell for improving delivery of the emitted light to the pharyngeal tissues.

2. The phototherapy device of claim 1, wherein:
the main body further includes an upper portion, a lower portion, a connecting portion joining the upper portion and the lower portion, and a channel located between the upper portion and the lower portion;
the channel is shaped to receive a tongue when inserted into the oral cavity;
the upper portion is shaped to overlie the tongue when the tongue is received in the channel; and
the lower portion is shaped to underlie the tongue when the tongue is received in the channel.

3. The phototherapy device of claim 1, wherein the shell is a rigid shell configured to maintain the airgap by maintaining a separation between the shell and the light source.

4. The phototherapy device of claim 1:
wherein the main body includes lateral walls and a distal end located between the lateral walls;
further comprising a camera located along the distal end configured to image tissues of the oral cavity being illuminated by the emitted light when the main body is inserted into the oral cavity.

5. The phototherapy device of claim 1, wherein:
the shell includes lateral walls and a distal end located between the lateral walls; and
the distal end includes lensing configured to alter a trajectory of the emitted light, such that the pharyngeal tissues are illuminated by the emitted light.

6. The phototherapy device of claim 1, wherein:
the main body is articulable along a point of articulation to form an angle of articulation between a proximal plane of the main body and a distal plane of the main body, such that a portion of the emitted light is targeted onto the pharyngeal tissues;
the proximal plane extends between lateral walls of the main body, a proximal end of the main body, and the point of articulation; and
the distal plane extends between the lateral walls of the main body, a distal end of the main body, and the point of articulation.

7. The phototherapy device of claim 1, wherein:
the main body is articulable along a distal point of articulation and a proximal point of articulation, such that a portion of the emitted light is targeted onto the pharyngeal tissues
the distal point of articulation is configured to form a distal angle of articulation between a central plane of the main body and a distal plane of the main body;
the proximal point of articulation is configured to form a proximal angle of articulation between a central plane of the main body and a proximal plane of the main body;
the proximal plane extends between lateral walls of the main body, a proximal end of the main body, and the proximal point of articulation;
the central plane extends between the lateral walls of the main body, the distal point of articulation, and the proximal point of articulation; and
the distal plane extends between the lateral walls of the main body, a distal end of the main body, and the distal point of articulation.

8. The phototherapy device of claim 1, wherein the light source includes light emitters for generating the emitted light.

9. The phototherapy device of claim 8, wherein:
the light emitters include distal light emitters and lateral light emitters;
the distal light emitters are positioned along a distal end of the main body, such that light emitted by the distal light emitters illuminates the pharyngeal tissues when the main body is positioned within the oral cavity; and
the lateral light emitters are positioned along lateral walls of the main body, such that light emitted by the lateral light emitters illuminates buccal tissues of the oral cavity when the main body is positioned within the oral cavity.

10. The phototherapy device of claim 1, wherein:
the main body includes a proximal end having a top bite location located along a top surface of the main body, and a bottom bite location located along a bottom surface of the main body;
the top bite location is configured to interact with top teeth of the oral cavity when the main body is located within the oral cavity; and
the bottom bite location is configured to interact with bottom teeth of the oral cavity when the main body is located within the oral cavity.

11. The phototherapy device of claim 10, wherein light emitted from the light source via the lateral walls of the main body is configured to illuminate buccal tissues of the oral cavity via an opening between the upper teeth and the lower teeth formed by the main body portion being located between the upper teeth and the bottom teeth of the oral cavity.

12. The phototherapy device of claim 1, further comprising side wings located adjacent the lateral walls of the main body, wherein:
the side wings each include a light emitting surface configured to emit light;
when the main body is located within the oral cavity the side wings are positioned relative to the main body, such that:
the side wings are located between teeth and buccal tissues of the oral cavity;
the light emitting surface of the side wings faces the buccal tissues; and
the light emitted from the light emitting surfaces illuminates the buccal tissues.

13. The phototherapy device of claim 1, wherein the main body includes a breathing channel comprising an opening extending between the distal end and the proximal end of the main body, such that when positioned within the oral cavity the breathing channels extends between the oral cavity and an external environment allowing for exchange of air between the oral cavity and the external environment.

14. The phototherapy device of claim 1, further comprising a breathing apparatus mechanically supported by an exterior surface of the main body, wherein:
the breathing channel includes an oral cavity opening, an external environment opening, and a breathing channel extending between the oral cavity opening and the external environment opening, such that when positioned within the oral cavity the breathing channel extends between the oral cavity and an external environment allowing for exchange of air between the oral cavity and the external environment.

15. The phototherapy device of claim 1, wherein the main body includes a protrusion extending from a top surface of the main body, such that:
the protrusion interacts with a roof of the oral cavity when the main body is positioned within the oral cavity; and
a gag reflex caused by the main body being positioned within the oral cavity is reduced;

16. The phototherapy device of claim 1, wherein the main body includes tongue protrusions extending from a bottom surface of the main body, such that when the main body is positioned within the oral cavity the tongue protrusions depress a tongue of the oral cavity.

17. The phototherapy device of claim 1, wherein the main body comprises a memory forming material configured to conform to a shape of the oral cavity when positioned within the oral cavity.

18. The phototherapy device of claim 1, wherein:
the main body includes location features comprising irregularities in a surface of the main body;
the location features are configured to, when the main body is located within the oral cavity:
engage with at least one of the teeth, lips, or palate; and
ensure that the main body is placed in a particular position and orientation within the oral cavity when the location features are positioned adjacent the corresponding structures of the oral cavity.

19. The phototherapy device of claim 1, further comprising processor circuitry mechanically supported by the main body, wherein the processor circuitry is configured to control emission of the light by the light source.

20. A phototherapy device for delivering phototherapy to an oral cavity, the phototherapy device including:
a main body configured to be received within the oral cavity and including an upper portion, a lower portion, a connecting portion joining the upper portion and the lower portion, and a channel located between the upper portion and the lower portion, wherein:
the channel is shaped to receive a tongue when inserted into the oral cavity;
the upper portion is shaped to overlie the tongue when the tongue is received in the channel; and
the lower portion is shaped to underlie the tongue when the tongue is received in the channel; and
a light source configured to emit light and mechanically supported by the main body, such that when the main body is positioned within the oral cavity the light source is located within the oral cavity and the emitted light illuminates pharyngeal tissues of the oral cavity including a dorsum of the tongue via light emitted from the upper portion, a ventral surface of the tongue via light emitted from the lower portion.
